(19) United States
(12) Patent Application Publication
(10) Pub. No.: US 2022/0088068 A1
(43) Pub. Date: Mar. 24, 2022

(54) CATALYTIC SYSTEMS INCLUDING A PEROXIDE FOR DELIVERY OF OXYGEN TO SKIN AND RELATED METHODS OF USE

(71) Applicant: L'Oreal, Paris (FR)

(72) Inventors: Anne-Laure Suzanne Bernard, New York, NY (US); Rebecca Chen, Princeton, NJ (US); William Bickford, Scotch Plains, NJ (US)

(21) Appl. No.: 17/543,503

(22) Filed: Dec. 6, 2021

Related U.S. Application Data
(62) Division of application No. 16/239,422, filed on Jan. 3, 2019.

(51) Int. Cl. A61K 33/40; A61K 9/00; A61K 9/06; A61K 47/02; A61K 8/22; A61K 8/02; A61K 8/04; A61Q 19/00; A61P 17/02

(52) U.S. Cl. CPC A61K 33/40; A61K 9/0002; A61K 9/009; A61K 9/0014; A61K 9/06; A61K 47/02; A61K 2800/874; A61K 8/0212; A61K 8/042; A61Q 19/00; A61P 17/02; A61K 2800/805; A61K 2800/88; A61K 8/22

(57) ABSTRACT

Systems, applicators, and methods for producing oxygen in a skincare formulation are described. In one embodiment, a system includes a skincare formulation comprising a peroxide; and an applicator that carries a peroxide-decomposing catalyst. The peroxide-decomposing catalyst is configured to come in contact with the peroxide and decompose the peroxide to provide oxygen gas proximate to a skin of a user.

### CROSS REFERENCE TO RELATED APPLICATION

This application is a divisional application of U.S. patent application Ser. No. 16/239,422 filed Jan. 3, 2019, the contents of which are incorporated herein by reference in their entirety.

### SUMMARY

In one aspect, the present disclosure provides system generally including a skincare formulation comprising a peroxide; and an applicator including a peroxide-decomposing catalyst, wherein the peroxide-decomposing catalyst is configured to decompose the peroxide to provide oxygen gas.

In accordance with any of the embodiments disclosed herein, the peroxide-decomposing catalyst maybe coupled to an application surface of the applicator. In accordance with any of the embodiments disclosed herein, the application surface may be a surface of a mask configured to conform to a face of a wearer. In accordance with any of the embodiments disclosed herein, the application surface may be selected from the group consisting of a bristle surface of a brush, a roller surface of a rolling applicator, and a blade surface of a spatula. In accordance with any of the embodiments disclosed herein, the skincare formulation may be disposed in a first compartment of a sachet and the application surface may be a surface of a second compartment of the sachet, and wherein the first compartment and the second compartment are fluidically separated by a breakable barrier.

In accordance with any of the embodiments disclosed herein, the peroxide-decomposing catalyst may coupled to the application surface with a binder. In accordance with any of the embodiments disclosed herein, the binder may be selected from the group consisting of latexes, polyacrylates, elastomers, hydrogels, and combinations thereof.

In accordance with any of the embodiments disclosed herein, the applicator includes a crosslinked hydrogel including the peroxide catalyst dispersed therein. In accordance with any of the embodiments disclosed herein, the crosslinked hydrogel may be freestanding. In accordance with any of the embodiments disclosed herein, the crosslinked hydrogel may be in a shape of a mask configured to conform to a face of the user. In accordance with any of the embodiments disclosed herein, the crosslinked hydrogel may be in a shape of one or more bristles of a brush.

In accordance with any of the embodiments disclosed herein, the peroxide-decomposing catalyst may be selected from the group consisting of iron oxide and a transition metal-based catalyst; an iron salt; sodium tungstate dehydrate, phosphoric acid, and a phase-transfer catalyst comprising a quaternary ammonium hydrogen sulfate; manganese dioxide; sodium bicarbonate and manganese dioxide; and a manganese salts.

In accordance with any of the embodiments disclosed herein, the skincare formulation further comprises an additive selected from the group consisting of a clay, an alkaline agent, a humectant, an antioxidant, and combinations thereof.

In accordance with any of the embodiments disclosed herein, the peroxide may be selected from the group consisting of hydrogen peroxide, urea peroxide, carbamide peroxide, calcium peroxide, magnesium peroxide, barium peroxide, zinc peroxide, lithium peroxide, dibenzoyl peroxide, peracetic acid, sodium peracetate, and combinations thereof.

In accordance with any of the embodiments disclosed herein, a concentration of the peroxide in the skincare formulation may be between about 0.1 wt. % and about 4 wt. %.

In accordance with any of the embodiments disclosed herein, the skincare formulation may be selected from the group consisting of an emulsion, a paste, a gel, and combinations thereof.

In another aspect, the present disclosure provides a method of contacting a portion of skin with oxygen gas generally including contacting a skincare formulation including a peroxide with an applicator including a peroxide-decomposing catalyst, thereby generating oxygen gas in the skincare formulation; and contacting the portion of skin with the skincare formulation with the applicator. In accordance with any of the embodiments disclosed herein, the peroxide-decomposing catalyst may be coupled to an application surface of the applicator, and wherein contacting the skincare formulation with the peroxide-decomposing catalyst includes contacting the skincare formulation with the application surface of the applicator.

In accordance with any of the embodiments disclosed herein, an oxygen concentration in the skincare formulation 1 minute after contacting the skincare formulation with the peroxide-decomposing catalyst may be in a range of about 15 mg/L to about 100 mg/L.

In accordance with any of the embodiments disclosed herein, contacting the portion of skin with the skincare formulation may include contacting the portion of skin with the skincare formulation for a duration of between about 1 minute and about 20 minutes.

This foregoing summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

FIG. 1 is a plan view of a mask applicator, in accordance with an embodiment of the disclosure.

FIG. 2 is a perspective view of a system including another applicator, in accordance with an embodiment of the disclosure.

FIG. 3 is a perspective view of another system including yet another applicator, in accordance with an embodiment of the disclosure.

FIG. 4 is a perspective view of another applicator, in accordance with an embodiment of the disclosure.

FIG. 5 is a perspective view of an applicator, in accordance with an embodiment of the disclosure.

FIG. 6A is a top-down plan view of a system, in accordance with an embodiment of the disclosure.

FIG. 6B is a perspective view of the system of FIG. 6A.

FIG. 7 schematically illustrates a method, in accordance with an embodiment of the disclosure.

Aspects and many of the attendant advantages of the claimed subject matter will become more readily appreciated as the same become better understood by reference to the following detailed description, when taken in conjunction with the accompanying drawings.

The detailed description set forth below in connection with the appended drawings, where like numerals reference like elements, is intended as a description of various embodiments of the disclosed subject matter and is not intended to represent the only embodiments. The embodiments described in this disclosure are provided merely as examples or illustrations and should not be construed as preferred or advantageous over other embodiments. The illustrative examples provided herein are not intended to be exhaustive or to limit the claimed subject matter to the exact forms disclosed.

### DETAILED DESCRIPTION

The following discussion provides examples of systems and methods suitable for contacting a portion of skin with oxygen gas. Several examples discussed below include systems including skincare formulation comprising hydrogen peroxide and a hydrogen peroxide-decomposing catalyst. As discussed in greater detail herein, such hydrogen peroxide-decomposing catalysts are configured to decompose hydrogen peroxide to generate oxygen gas. When the skincare formulation is applied to portion of skin, for example, with the surface including the hydrogen peroxide-decomposing catalyst the portion of skin is exposed to heightened levels of oxygen.

Oxygen therapy including exposing skin to oxygen is believed to have therapeutic benefits including, for example, enhanced wound healing, skin brightening, skin detoxification, and skin repair. Conventional methods of oxygen delivery, such as compressed oxygen gas or exposing hydrogen peroxide to alkaline environments, generally result in low levels of oxygen that generate oxygen slowly. Such low levels of oxygen may be insufficient to provide certain benefits associated with providing oxygen therapy to skin and wounds.

Toward that end, in certain aspects the present disclosure provides systems and methods for providing oxygen to a portion of skin. In certain embodiments, the systems of the present disclosure include a skincare formulation comprising a peroxide; and an applicator including a peroxide-decomposing catalyst. As discussed further herein, the peroxide-decomposing catalyst is configured to decompose the peroxide to provide oxygen gas. In this regard, by contacting the skincare formulation including a peroxide with the applicator including a peroxide-decomposing catalyst oxygen gas is generated and is dissolved or otherwise dispersed in the skincare formulation. The portion of skin may be contacted with the skincare formulation containing oxygen, such as with the applicator, thereby delivering high levels of oxygen to the portion of skin.

In the following description, numerous specific details are set forth in order to provide a thorough understanding of one or more embodiments of the present disclosure. It will be apparent to one skilled in the art, however, that many embodiments of the present disclosure may be practiced without some or all of the specific details. Further, it will be appreciated that embodiments of the present disclosure may employ any combination of features described herein.

In an aspect, the present disclosure provides a system for delivery of oxygen to a portion of skin. In an embodiment, the system includes a skincare formulation comprising a peroxide; and an applicator including a peroxide-decomposing catalyst, wherein the peroxide-decomposing catalyst is configured to decompose the peroxide to provide oxygen gas. In an embodiment, the applicators of the present disclosure are provided separately from the skincare formulations described herein.

In an embodiment, the peroxide-decomposing catalyst is coupled to an application surface of the applicator. In an embodiment, the peroxide-decomposing catalyst is coupled to the application surface with a binder. In this regard, the peroxide-decomposing catalyst is on the application surface and configured to contact the skincare formulation and to apply skincare formulation that has contacted the peroxide-decomposing catalyst. In an embodiment, the binder is selected from the group consisting of latexes, polyacrylates, elastomers, hydrogels, and combinations thereof. In an embodiment, the application surface includes a plurality of fibers coated with the peroxide-decomposing catalyst. In an embodiment, the plurality of fibers are cast or otherwise formed into a non-woven fabric, such as in the shape of an applicator.

In an embodiment, the applicator includes a gel, such as a crosslinked hydrogel, and the peroxide-decomposing catalyst is disposed within the gel. Such gels may be prepared by, for example, crosslinking a mixture including gel precursors and the peroxide-decomposing catalyst such that the peroxide-decomposing catalyst is disposed therein. By crosslinking the gel, the peroxide-decomposing catalyst is fixed within the crosslinked matrix. In this regard, the gel including the peroxide-decomposing catalyst is configured to decompose peroxide contained in skincare formulations described herein to provide oxygen gas when such skincare formulations are contacted with the gel. In an embodiment, the gel has sufficient structural integrity, such as through a sufficiently high level of crosslinking, that it is free-standing. In an embodiment, the gel has sufficient structural integrity that the applicator including the gel is not coupled to a separate structure, such as a structure to provide additional rigidity to the applicator.

By coupling the peroxide-decomposing catalyst to the applicator, such as by coupling the peroxide-decomposing catalyst to an application surface and/or fixing the peroxide-decomposing catalyst in a crosslinked gel, the applicators of the present disclosure may be used two or more times to decompose peroxide in a skincare formulation. In this regard, the applicators of the present disclosure and the skincare formulations of the present disclosure may be sold separately from one another. Further, by using a heterogeneous peroxide-decomposing catalyst coupled to an application surface and/or fixed in a crosslinked gel, a user may selectively contact a skincare formulation with the peroxide-decomposing catalyst. This is in contrast to skincare formulations in which a peroxide-decomposing catalyst is dissolved or dispersed, such as on particles dispersed therein. In such a configuration, peroxide in the skincare formulation is depleted, such as during shipment and storage, and, consequently, oxygen is also generated and depleted constantly, thus ultimately providing less oxygen to a user.

In an embodiment, the applicator is a single-layer or single-sheet applicator on which the peroxide-composing catalyst is fixedly coupled. This is in contrast to a system including two or more layers or sheets that are movably independent of one another.

The systems of the present disclosure include a peroxide-decomposing catalyst. The peroxide-decomposing catalyst can be configured to decompose peroxide in a skincare formulation to provide oxygen dispersed or dissolved therein. In an embodiment, the peroxide-decomposing catalyst includes iron oxide and a transition metal-based catalyst. In certain further embodiments, the peroxide-decomposing catalyst further includes an iron salt. In an embodiment, the iron salt is selected from the group consisting of ferrous sulfate, ferrous gluconate, ferric chloride, and combinations thereof. In an embodiment, the peroxide-decomposing catalyst includes sodium tungstate dehydrate, phosphoric acid, and a phase-transfer catalyst comprising a quaternary ammonium hydrogen sulfate. In an embodiment, the peroxide-decomposing catalyst includes manganese dioxide. In an embodiment, the peroxide-decomposing catalyst includes sodium bicarbonate, manganese dioxide, and a manganese salt. In an embodiment, the manganese salt is selected from the group consisting of manganese gluconate, manganese PCA, manganese chloride, and combinations thereof.

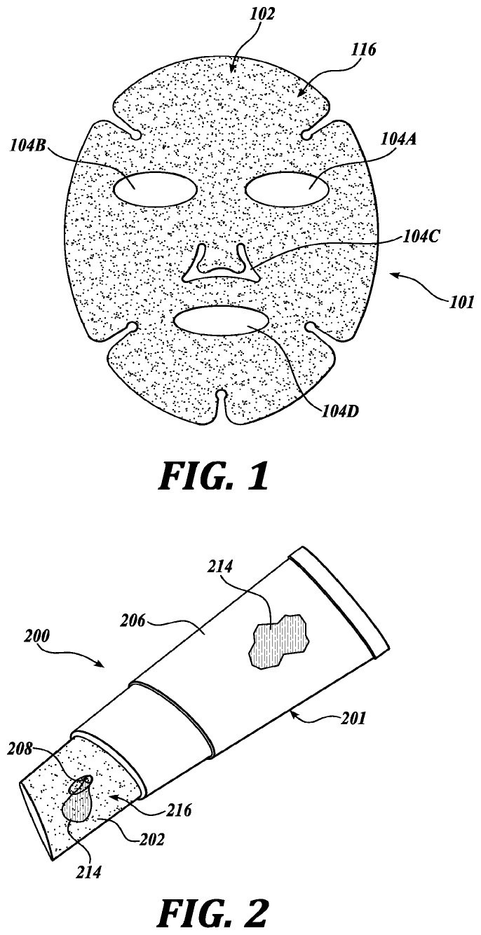

Turning now to FIG. 1 there is shown an example of an applicator 101 in accordance with embodiments of the present disclosure. In the illustrated embodiment, applicator 101 is in the form of a mask configured to conform to the face of a user and includes apertures 104A-104D, application surface 102, and peroxide-decomposing catalyst 116. In this regard, a skincare formulation (not shown, see FIGS. 2, 3, and 6) as described herein may be contacted with application surface 102, thereby decomposing peroxide in the skincare formulation to provide oxygen. The skincare formulation now containing oxygen dissolved or otherwise dispersed therein may be applied to, for example, a face of a user with the mask 101 thereby contacting the skin of the face with oxygen. As discussed elsewhere herein, in an embodiment, the peroxide-decomposing catalyst 116 is coupled to application surface 102 such as with a binder. In other embodiments, the mask 101 comprises a gel including peroxide-decomposing catalyst 116 dispersed therein and, for example, does not include a surface to which the gel is coupled.

In an embodiment, the systems of the present disclosure include an applicator having an application surface in the form of a slider 200, for example, a spatula. In that regard, attention is directed to FIG. 2 in which a system 200 in accordance with an embodiment of the disclosure is illustrated. As shown, system 200 includes applicator 201 having an applicator surface 202 including a peroxide-decomposing catalyst 216. In an embodiment, skincare formulation 214 is disposed in a portion of the applicator 201. In the illustrated embodiment, applicator surface 202 further includes an aperture 208 through which skincare formulation 214 disposed in handle 206 may be extruded, such as through application of pressure to handle 206 by a user. In this regard, a user may contact the skincare formulation 214 with the catalytic application surface 202, thereby generating oxygen in the skincare formulation 214, before applying the skincare formulation 214 to a portion of skin.

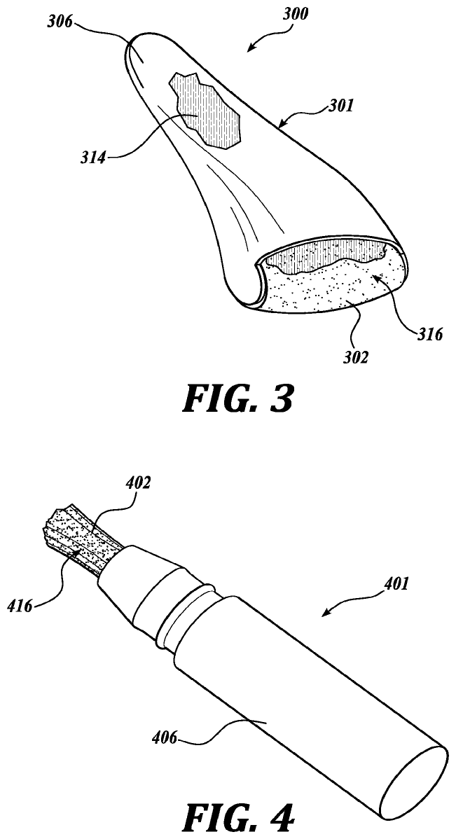

In another embodiment, the systems of the present disclosure include an applicator in the form of a rolling applicator and the application surface is a rolling application surface. In that regard, attention is directed to FIG. 3 in which system 300 including rolling applicator 301 in accordance with an embodiment of the disclosure is illustrated. Rolling applicator 301 is shown to include a handle 306 and rolling applicator surface 302 including a peroxide-decomposing catalyst 316. Rolling applicator surface 302 is configured to roll, such as when rolling applicator surface 302 is applied to a portion of skin. In that regard, a user may contact rolling applicator surface 302 with a skincare formulation 314 as described further herein and apply the skincare formulation 314 to a portion of skin, for example, after a duration sufficient to generate oxygen in the skincare formulation 314. In the illustrated embodiment, skincare formulation 314 is disposed in a portion of handle 306 such that it may be placed in contact with the rolling applicator surface 302, such as by a user applying pressure to handle 306.

In an embodiment, the applicators of the present disclosure are in the form of a brush and the application surface includes a surface of a plurality of bristles. In that regard, attention is directed to FIG. 4 in which an applicator brush 401 in accordance with embodiments of the present disclosure is illustrated. As shown, applicator brush 401 includes a handle 406 configured, for example, to be held by the hand of a user and a plurality of bristles 402. In an embodiment, the peroxide-decomposing catalyst 416 is coupled to a surface of the plurality of bristles 402, as described further herein. In another embodiment, the plurality of bristles 402 includes a crosslinked gel in which the peroxide-decomposition catalyst 416 is disposed. In this regard, oxygen is generated when a skincare formulation (not shown, see for example FIGS. 2, 3, and 6) including a peroxide as described herein is contacted with the plurality of bristles 402. Further, the applicator brush 401 is suitable to apply the skincare formulation now containing oxygen dissolved or otherwise dispersed therein to a portion of skin, thereby providing oxygen to the portion of skin.

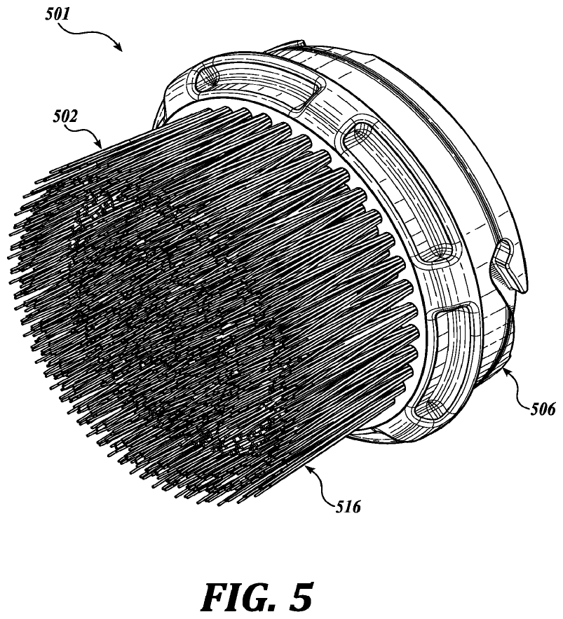

In an embodiment, the applicators of the present disclosure are in the form of an end effector, such as a brush, coupleable to an appliance and configured to receive motion from the appliance. In that regard, attention is directed to FIG. 5 in which an applicator 501 in accordance with an embodiment of the disclosure is illustrated. As shown, end effector 501 includes a base portion 506 and a plurality of bristles 502 coupled to the base portion 506. While end effector 501 including the plurality of bristles 502 is shown, it will be understood that end effectors including other contact members, such as exfoliators, applicators, and the like, are encompassed by the present disclosure. Base portion 506 is coupleable to an appliance (not shown) and to receive motion from the appliance, such as from a motor disposed in the appliance. In an embodiment, the peroxide-decomposing catalyst 516 is coupled to a surface of the plurality of bristles 502, as described further herein. In another embodiment, the plurality of bristles 502 includes a crosslinked gel in which the peroxide-decomposing catalyst 516 is disposed. In this regard, oxygen is generated when a skincare formulation (not shown, see for example FIGS. 2, 3, and 6) including a peroxide as described herein is contacted with the plurality of bristles 502. Further, the skincare formulation now containing oxygen dissolved or otherwise dispersed therein may be applied to a portion of skin with end effector 501, such as when coupled to and receiving motion from an appliance.

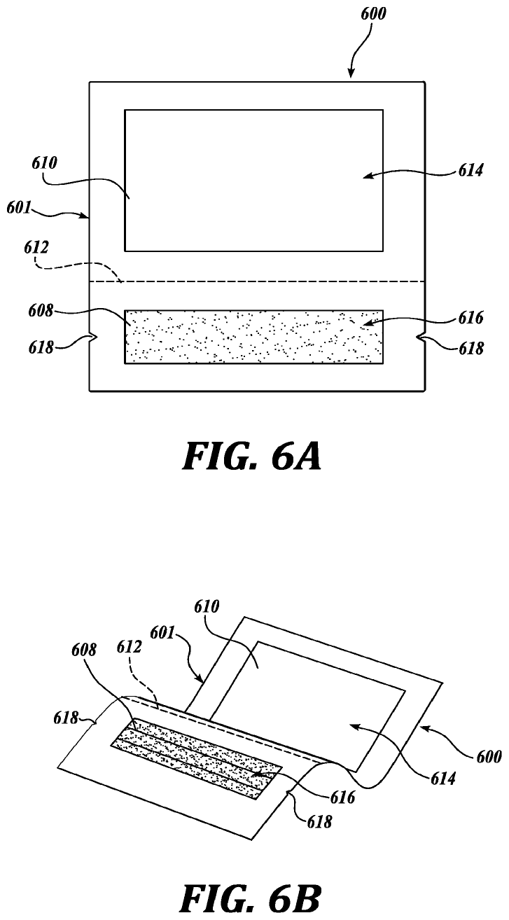

In an embodiment, the systems of the present disclosure include a sachet in which the skincare formulation and peroxide-decomposing catalyst are disposed, wherein the skincare formulation and the peroxide-decomposing catalyst are separated by a breakable barrier. In that regard, attention is directed to FIGS. 6A and 6B in which a system 600 in accordance with embodiments of the present disclosure is illustrated. FIG. 6A is a top-down plan view of system 600. FIG. 6B is a perspective view of the system 600 of FIG. 6A. As shown, system 600 is in the form of a sachet (or other container) 601 including a first compartment (also referred to as a pouch) 610 in which the skincare formulation 614 including a peroxide is disposed and a second compartment (also referred to as a pouch) 608 in which the peroxide-decomposing catalyst 616 is disposed. The first pouch 610 and the second pouch 608 are separated by a breakable barrier 612. Although the barrier 612 is described as the breakable barrier, in some embodiments, the barrier 612 is a removable or a dissolvable barrier. In an initial state in which breakable barrier 612 is intact, the first pouch 610 and the second pouch 608 are separated. When breakable barrier 612 is intact, skincare formulation 614 and peroxide-decomposing catalyst 616 are kept apart. In this regard, skincare formulation 614 and peroxide-decomposing catalyst 616 do not generate oxygen during shipment or storage that may dissipate prior to breaking the breakable barrier 612. Rather, skincare formulation 614 and peroxide-decomposing catalyst 616 are in contact and thus generate oxygen in the skincare formulation 614 when breakable barrier 612 is broken or removed, such as by a user. Accordingly, system 600 is configured to provide skincare formulation 614 having oxygen concentrations that are generally higher when applied to a portion of skin than if skincare formulation 614 were, for example, shipped in contact with peroxide-decomposing catalyst 616.

After skincare formulation 614 has been in contact with peroxide-decomposing catalyst 616 for a time sufficient to generate oxygen in the skincare formulation 614, system 600 may be used to apply skincare formulation 614 to a portion of skin. In the illustrated embodiment, sachet 601 includes notches 618 disposed at an outer edge of sachet 601 and adjacent to second pouch 608. In this regard, a user may tear open second pouch 608, for example, beginning at notches 618 when skincare formulation 614 has been in contact with the peroxide-decomposing catalyst 616 for a time sufficient to generate oxygen, so that the skincare formulation 614 and oxygen dissolved or dispersed therein may be applied to a portion of skin.

The systems of the present disclosure include skincare formulations including a peroxide dissolved or otherwise dispersed therein. As discussed further herein, the peroxides present in the skincare formulations react with the peroxide-decomposing catalysts of the applicators described further herein to decompose and provide oxygen. The peroxide can be any peroxide suitable to decompose when contacted with a catalyst to provide oxygen in the skincare formulation. In an embodiment, the peroxide is selected from the group consisting of hydrogen peroxide, urea peroxide, carbamide peroxide, calcium peroxide, magnesium peroxide, barium peroxide, zinc peroxide, lithium peroxide, dibenzoyl peroxide, peracetic acid, sodium peracetate, and combinations thereof.

In an embodiment, a concentration of the peroxide in the skincare formulation is in a range of about 0.1 wt. % and about 4 wt. %. In an embodiment, the concentration of the peroxide in the skincare formulation is in a range of about 0.2 wt. % and about 2 wt. %. In an embodiment, the concentration of the peroxide in the skincare formulation is in a range of about 0.2 wt. % and about 1 wt. %. Such concentrations are generally sufficient to provide high levels of oxygen in the skincare formulations after contact with the peroxide-decomposing catalyst and provide the benefits of oxygen therapy, as discussed further herein.

The skincare formulations described herein include any skincare formulation including a peroxide. Such skincare formulations can include, for example, gels, pastes, oil/water emulsions, water/oil emulsions, foams, and the like. In an embodiment, the skincare formulation is selected from the group consisting of a moisturizer, an exfoliant, a cleaning formulation, a sunscreen, an antibiotic ointment, and combinations thereof.

In an embodiment, the skincare formulation further includes one or more additives. In an embodiment, the additive is selected from the group consisting of a clay, an alkaline agent, a humectant, an antioxidant, and combinations thereof. In certain embodiments, the clay and alkaline agents each accelerate decomposition reactions of the peroxide, thus providing increased amounts of oxygen in skincare formulations contacted by a peroxide-decomposing catalyst. In an embodiment, the clay includes zeolites. Without wishing to be bound by theory, it is believed that the zeolites accelerate decomposition reactions between peroxide-decomposing catalysts and the peroxides. In an embodiment, the alkaline agent is selected from the group consisting of alkali metal hydroxides (e.g., lithium hydroxide, sodium hydroxide, potassium hydroxide, and magnesium hydroxide), alkaline-earth metal hydroxides (e.g., calcium hydroxide), alkali metal silicates, (e.g., sodium silicate, lithium silicate, potassium silicate, magnesium silicate, aluminum silicate, and fluorphlogopite), alkali metal carbonates (e.g., lithium carbonate, sodium carbonate, potassium carbonate, and sodium bicarbonate), alkaline-earth metal carbonates (e.g., calcium carbonate), organic carbonates (e.g., guanidine carbonate), basic amino acids (arginine, lysine, and histidine), polymeric forms of basic amino acids (e.g. poly(arginine) and poly(lysine)), organic amines such as alkanolamines (e.g., monoethanolamine, diethanolamine, triethanolamine, and aminomethyl propanol), ammonium hydroxide, and combinations thereof.

Humectants and antioxidants are typically beneficial to the skin of a user. Furthermore, antioxidants are typically suitable to decrease an amount of reactive oxygen species (ROS), such as in the skincare formulation. In certain embodiments, the oxygen provided by decomposed peroxides generates ROS, which may be harmful to users. By decreasing the amount of ROS present in the skincare formulation with antioxidants a health benefit may be conferred to the user in addition to the benefits associated with oxygen therapy described further herein.

In another aspect, the present disclosure provides a method of contacting a portion of skin with oxygen gas. In an embodiment, the method includes contacting a skincare formulation including a peroxide with an applicator including a peroxide-decomposing catalyst, thereby generating oxygen gas in the skincare formulation; and contacting the portion of skin with the skincare formulation with the applicator.

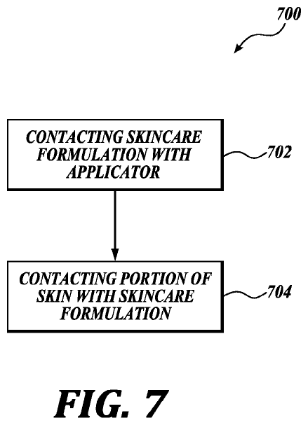

In that regard, attention is directed FIG. 7, in which a method 700 according to embodiments of the present disclosure is schematically illustrated.

Method 700 may begin with process block 702, which includes contacting the skincare formulation with an applicator. In an embodiment, contacting the skincare formulation with an applicator includes contacting the skincare formulation with an applicator as described further herein, such as with respect to FIGS. 1-6B. In an embodiment, the applicator is selected from applicators 101, 201, 301, 401, 501, and 601. In some embodiments, peroxides in the skincare formulation decompose only when in contact with the peroxide-decomposing catalyst. In this regard, applying the skincare formulation to a portion of skin with the applicator, such as with a brush, end effector, rolling applicator, spatula, and the like, for an extended period of time provides greater oxygen levels in the skincare formulation.

In an embodiment, contacting the skincare formulation with the applicator includes contacting the skincare formulation with the applicator for a time sufficient to decompose the peroxide in the skincare formulation thereby generating oxygen. In an embodiment, the time sufficient to decompose the peroxide in the skincare formulation thereby generating oxygen is a time in a range between 30 seconds and 20 minutes. In an embodiment, the time sufficient to decompose the peroxide in the skincare formulation thereby generating oxygen is a time in a range between 1 minute and 10 minutes. In an embodiment, the time sufficient to decompose the peroxide in the skincare formulation thereby generating oxygen is a time in a range between 1 minute and 5 minutes.

In an embodiment, an oxygen concentration in the skincare formulation 1 minute after contacting the skincare formulation with the peroxide-decomposing catalyst is in a range of about 15 mg/L to about 100 mg/L. In an embodiment, an oxygen concentration in the skincare formulation 1 minute after contacting the skincare formulation with the peroxide-decomposing catalyst is in a range of about 20 mg/L to about 75 mg/L. In an embodiment, an oxygen concentration in the skincare formulation 1 minute after contacting the skincare formulation with the peroxide-decomposing catalyst is in a range of about 30 mg/L to about 50 mg/L.

In an embodiment, a concentration of the peroxide in the skincare formulation is in a range of about 0.1 wt. % and about 4 wt. %. In an embodiment, the concentration of the peroxide in the skincare formulation is in a range of about 0.2 wt. % and about 2 wt. %. In an embodiment, the concentration of the peroxide in the skincare formulation is in a range of about 0.2 wt. % and about 1 wt. %.

In an embodiment, the skincare formulation is any skincare formulation described herein. In an embodiment, the skincare formulation includes an additive selected from the group consisting of a clay, an alkaline agent, a humectant, an antioxidant, and combinations thereof. In an embodiment, the peroxide is selected from the group consisting of hydrogen peroxide, urea peroxide, carbamide peroxide, calcium peroxide, magnesium peroxide, barium peroxide, zinc peroxide, lithium peroxide, dibenzoyl peroxide, peracetic acid, sodium peracetate, and combinations thereof.

In an embodiment, process block 702 is followed by process block 704, which includes contacting a portion of skin with skincare formulation. As described further herein, by contacting the skincare formulation containing a peroxide with the applicators described herein including a peroxide-decomposing catalyst, oxygen is generated in the skincare formulation. In an embodiment, the skincare formulation is contacted by the applicator prior to the skincare formulation contacting the portion of skin. In an embodiment, the skincare formulation contacts the applicator and the portion of skin simultaneously. In an embodiment, the skincare formulation is contacted with the portion of skin prior to contacting the skincare formulation with the applicator. In any event, by contacting the portion of skin with the skincare formulation and contacting the skincare formulation with the applicator, the portion of skin is exposed to the oxygen in the skincare formulation, thereby providing the portion of skin with the benefits of oxygen therapy discussed further herein.

The order in which some or all of the process blocks appear in method 700 should not be deemed limiting. Rather, one of ordinary skill in the art having the benefit of the present disclosure will understand that some of the process blocks may be executed in a variety of orders not illustrated, or even in parallel. As above, in some embodiments, process block 702 precedes process block 704. In some other embodiments, process block 704 precedes process block 702.

It should be noted that for purposes of this disclosure, terminology such as “upper,” “lower,” “vertical,” “horizontal,” “inwardly,” “outwardly,” “inner,” “outer,” “front,” “rear,” etc., should be construed as descriptive and not limiting the scope of the claimed subject matter. Further, the use of “including,” “comprising,” or “having” and variations thereof herein is meant to encompass the items listed thereafter and equivalents thereof as well as additional items. Unless limited otherwise, the terms “connected,” “coupled,” and “mounted” and variations thereof herein are used broadly and encompass direct and indirect connections, couplings, and mountings. The term “about” means plus or minus 5% of the stated value.

The principles, representative embodiments, and modes of operation of the present disclosure have been described in the foregoing description. However, aspects of the present disclosure which are intended to be protected are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

While illustrative embodiments have been illustrated and described, it will be appreciated that various changes can be made therein without departing from the spirit and scope of the invention.

The embodiments of the invention in which an exclusive property or privilege is claimed are defined as follows:

1. A system comprising:
a skin care formulation comprising a peroxide;
a peroxide-decomposing catalyst configured to decompose the peroxide to provide oxygen gas; and
an applicator including a handle and an application surface coupled with the handle, wherein the application surface is configured to contact the skin care formulation with the peroxide-decomposing catalyst as part of application of the skin care formulation to a portion of skin of a user.

2. The system of claim 1, wherein the application surface is disposed on a roller, a brush, or a spatula.

3. The system of claim 1, wherein the peroxide-decomposing catalyst is coupled with the application surface.

4. The system of claim 3, wherein the peroxide-decomposing catalyst is coupled to the application surface with a binder.

5. The system of claim 4, wherein the binder comprises a hydrogel.

6. The system of claim 1, wherein the skin care formulation is disposed in the handle.

7. The system of claim 6, wherein the handle is configured to provide the skin care formulation to the peroxide-decomposing catalyst on the application surface.

8. The system of claim 7, wherein the handle is configured to extrude the skin care formulation onto the application surface upon application of pressure to the handle.

9. The system of claim 7, wherein the applicator is configured to extrude the skin care formulation through an aperture disposed in the application surface.

10. The system of claim 1, wherein the applicator includes a crosslinked hydrogel including the peroxide catalyst dispersed therein.

11. The system of claim 10, wherein the crosslinked hydrogel is freestanding.

12. The system of claim 10, wherein the crosslinked hydrogel is in a shape of one or more bristles of a brush.

13. The system of claim 1, wherein the peroxide-decomposing catalyst is a transition metal salt.

14. The system of claim 13, wherein the peroxide-decomposing catalyst is sodium tungstate dehydrate.

15. The system of claim 1, wherein the skin care formulation further comprises an additive that reacts with reactive oxygen species to reduce an amount of reactive oxygen species in the skin care formulation.

16. The system of claim 15, wherein the additive comprises an antioxidant.

17. The system of claim 1, wherein the peroxide comprises hydrogen peroxide.

18. The system of claim 1, wherein a concentration of the peroxide in the skin care formulation is between about 0.1 wt. % and about 4 wt. %.

19. The system of claim 1, wherein the skin care formulation is selected from the group consisting of an emulsion, a paste, a gel, and combinations thereof.

20. The system of claim 1, wherein the skin care formulation comprises a moisturizer.
